(19) United States
(12) Patent Application Publication
(10) Pub. No.: US 2025/0098746 A1
(43) Pub. Date: Mar. 27, 2025

(54) AEROSOL GENERATION AND DELIVERY SYSTEM FOR CARBOXYLATED ACTIVES

(71) Applicant: Nicoventures Trading Limited, London (GB)

(72) Inventors: Oriol STROPHAIR, London (GB); Ashley DAVIES, London (GB)

(21) Appl. No.: 18/290,955

(22) PCT Filed: Jul. 21, 2022
(86) PCT No.: PCT/GB2022/051892

§ 371 Date: Jan. 22, 2024

(30) Foreign Application Priority Data
Jul. 22, 2021 (GB) 2110546.5

(51) Int. Cl. A24F 40/30; A24F 40/42; A24F 40/46; A61K 31/192

(52) U.S. Cl. CPC A24F 40/30; A24F 40/42; A24F 40/46; A61K 31/192

(57) ABSTRACT

A delivery system including a powered aerosol generating device and an aerosolizable material, wherein the powered aerosol generating device includes a power source and a controller, and the system includes a first aerosolizable material and a second aerosolizable material, wherein the second aerosolizable material includes at least one carboxylated active and is stored in the system separately from the first aerosolizable material.

### RELATED APPLICATIONS

The present application is a National Phase Entry of PCT Application No. PCT/GB2022/051892 filed Jul. 21, 2022, which claims priority to GB Application No. 2110546.5 filed Jul. 22, 2021, each of which is hereby incorporated by reference in their entirety.

### FIELD OF THE INVENTION

The present invention relates to a delivery system, as well as to components and aerosolizable materials for use in the system.

### BACKGROUND

Delivery systems that generate an aerosol for inhalation by a user are known in the art. In particular, non-combustible aerosol provision systems that generate an aerosol for inhalation by a user are known in the art. Such systems typically comprise an aerosol generator that is capable of converting an aerosolizable material into an aerosol. In some instances, the aerosol generated is a condensation aerosol whereby an aerosolizable material is first vaporized and subsequently allowed to condense into an aerosol. In other instances, the aerosol generated is an aerosol that results from the atomization of the aerosolizable material. Such atomization may be brought about mechanically, e.g. by subjecting the aerosolizable material to vibrations to form small particles of material that are entrained in airflow. Alternatively, such atomization may be brought about electrostatically, or in other ways, such as by using pressure etc.

The aerosolizable material typically contains a variety of components that are to be delivered to a user. Depending on the mode of action of the aerosol generator, these components may be influenced by the aerosol generator in different ways.

Ultimately, it is important to design such delivery systems and the associated aerosolizable material so that they provide for a positive user experience.

### SUMMARY OF THE INVENTION

The present invention relates to a delivery system comprising a powered aerosol generating device and an aerosolizable material, wherein the aerosolizable material comprises at least one carboxylated active, and wherein the system is configured to provide for selective decarboxylation of the carboxylated active.

The carboxylated form of some active materials, such as cannabinoids, may have a different stability profile compared to the decarboxylated form. For example, the carboxylated form of cannabidiol, cannabidiolic acid (CBDA), behaves differently in some solvent systems compared to the decarboxylated form (CBD). On the one hand, this difference in stability can be exploited since it is possible to deploy a particular form of the cannabinoid so as to achieve a desired stability profile. However, it is generally the case that cannabinoids exert a greater pharmacological effect in their decarboxylated form. Thus, providing a cannabinoid in its carboxylated form may be less desirable. It is, however, possible to convert cannabinoids from their carboxylated form to their decarboxylated form.

The present invention provides for the selective decarboxylation of the active, such as the cannabinoid, within the system. By “selective decarboxylation of the carboxylated active” it is meant that the system is able to selectively increase the extent to which decarboxylation of the carboxylated active takes place. This is advantageous, since it is possible to exploit the benefits of controlling the stability profile of the active, whilst also allowing for the provision of an aerosol with a decarboxylate quantity similar to that which might be derived from an aerosolizable material containing the decarboxylate form of the active only.

In one aspect, the present invention relates to a delivery system comprising a powered aerosol generating device and an aerosolizable material, wherein the powered aerosol generating device comprises a power source and a controller, and the system comprises a first aerosolizable material and a second aerosolizable material, wherein the second aerosolizable material comprises at least one carboxylated active and is stored in the system separately from the first aerosolizable material. For the avoidance of doubt, the use of this alternative approach can be combined with the other approaches described herein for the selective decarboxylation of the carboxylated active.

Locating the second aerosolizable material separately from the first aerosolizable material can be beneficial for a number of reasons. Firstly, it can allow for the second aerosolizable material to be subjected to selective heating to a temperature which is lower than the temperature to which the first aerosolizable material is heated. For example, the second aerosolizable material can be stored in a second reservoir which is separate from a reservoir in which the first aerosolizable material is located. In this way, the second reservoir could be selectively heated (via power from a power source in the device or elsewhere) so as to facilitate decarboxylation of the carboxylated active (e.g. cannabinoid) contained therein. One or more heaters can be provided to heat the second aerosolizable material. The second reservoir could contain an internal heater which would be in contact with the second aerosolizable material and/or an external heater which would not be in contact with the second aerosolizable material.

The extent to which the second reservoir is heated affects the extent of decarboxylation that may occur. For example, the heater (whether it be internal, external or both) is configured to heat the second aerosolizable material to a temperature above ambient, but below the temperature at which significant vaporization of the second aerosolizable material would take place. In this regard, the second aerosolizable material may be heated to a temperature such as greater than 50° C., greater than 60° C., greater than 70° C., greater than 80° C., greater than 90° C., greater than 100° C., greater than 110° C., greater than 120° C., greater than 130° C., greater than 140° C., or greater than 145° C. Preferably, the second aerosolizable material is not heated above 150° C. when in the second reservoir. Preferably, the second aerosolizable material is heated to a temperature of between 50° C. and 150° C., such as between 50° C. and 140° C., between 50° C. and 130° C., between 50° C. and 120° C., between 50° C. and 110° C., between 50° C. and 100° C., between 50° C. and 90° C., between 50° C. and 80° C., or between 60° C. and 150° C., between 70° C. and 80° C., between 90° C. and 150° C., between 100° C. and 150° C., between 110° C. and 150° C., between 120° C. and 150° C., between 130° C. and 150° C., between 140° C. and 150° C.

The second aerosolizable material may be heated, typically to one of the above mentioned temperatures, for a sustained period of time. For example, the second aerosolizable material may be heated to one of the above mentioned temperatures for more than 10 s, more than 20 s, more than 30 s, more than 40 s, more than 50 s, more than 60 s, more than 1 min, more than 2 min, more than 3 min, more than 4 min, more than 5 min, more than 10 min, more than 15 min, more than 20 min or more than 30 min. The total heating time may decrease with increasing temperature. In this regard, the user can select, via the controller, to heat the second aerosolizable material for a shorter period of time at a higher temperature, or for a longer period of time at a lower temperature. The precise temperature and length of heating can be determined by the user.

The first and second reservoirs are fluidly connected. This facilitates transfer of the second aerosolizable material to the first reservoir. The fluid connection could be any one of a wick, pump, membrane or the like. A membrane may be useful in that it may be used to permit passage of decarboxylated active from the second reservoir to the first reservoir. The user can control the extent to which transfer of the second aerosolizable material to the first reservoir occurs. For example, a pump could be manually operated by the user, or a powered pump could be controlled to transfer a particular quantity of second aerosolizable material when controlled to do so by the user. In this regard, one or more button, switches, touchpads or the like could be used to control the transfer of the second aerosolizable material to the first reservoir. Alternatively, this could be controlled via another device which is remotely connected (e.g. wirelessly) to the device of the delivery system. The fluid connection could also serve to decarboxylate the carboxylated active. For example, the fluid connection could be thermally coupled to one or more heaters. In this way, decarboxylated active can be fed directly to the first reservoir. This approach avoids having to subject the entire second reservoir (and the carboxylated active therein) to heat in order to decarboxylate, since only that fluid connection portion which is being heated is subject to decarboxylation.

Whilst this aspect of the invention is generally employed with two reservoirs, it is possible for there to only be a single reservoir containing a single aerosolizable material. In this situation, the single aerosolizable material contains the carboxylated active and it is subjected to heat from a dedicated internal and/or external heater (as described above), or it is possible that heat generated by the aerosol generator can be used.

Another reason why it can be beneficial to store the second aerosolizable material separately from the first aerosolizable material (in respective reservoirs) is that it is possible to stably store the active (such as the cannabinoid) at higher, sometimes much higher concentrations in the carboxylated form than might be possible for the decarboxylated form. As a result, it is possible to use the aerosolizable material having a high concentration of carboxylated active to replenish the first aerosolizable material. This means that it is possible for the user to “boost” or simply replenish the carboxylated active content within the first aerosolizable material as they wish. Due to the ability to exploit the in situ decarboxylation which can be facilitated during aerosolization, it is thus possible for users to stably store aerosolizable materials having relatively high concentrations of carboxylated active yet be able to dose such materials into the first aerosolizable material just prior to use. Accordingly, whilst heating the second aerosolizable material in the second reservoir can facilitate pre-emptive decarboxylation, this is not required. This approach is particular suitable where the actives are cannabinoids.

As explained above, the first and second reservoirs are fluidly connected. This facilitates transfer of the second aerosolizable material to the first reservoir. The fluid connection could be any one of a wick, pump, membrane or the like. The user can control the extent to which transfer of the second aerosolizable material to the first reservoir occurs. For example, a pump could be manually operated by the user, or a powdered pump could be controlled to transfer a particular quantity of second aerosolizable material when controlled to do so by the user. In this regard, one or more button, switches, touchpads or the like could be used to control the transfer of the second aerosolizable material to the first reservoir. Alternatively, this could be controlled via another device which is remotely connected (e.g. wirelessly) to the device of the delivery system.

It is also possible for the controller to be programmed such that it initiates transfer of a particular quantity of second aerosolizable material according to a particular schedule. For example, it may be possible for a specific quantity of second aerosolizable material, e.g. 0.1 ml, 0.2 ml, 0.3 ml, 0.4 ml, 0.5 ml, 0.6 ml, 0.7 ml, 0.8 ml, 0.9 ml, or 1 ml, to be transferred on an hourly, daily, weekly or monthly schedule.

It is also possible for the controller to control transfer of the second aerosolizable material in dependence on the aerosolization of the first aerosolizable material. For example, the controller can be configured to monitor the instances and/or duration (or combination thereof) of power supplied to aerosolize the first aerosolizable material and to transfer a related proportion of second aerosolizable material. In this regard, for a particular system, device, aerosolizable material and power setting, it is possible to empirically determine the mass loss resulting from aerosolization of the first aerosolizable material. This mass loss can be used to define the related proportion of the second aerosolizable material that is transferred. It will be understood that once the second aerosolizable material is transferred to the first reservoir, it becomes part of the first aerosolizable material.

It is also possible (in addition to any one or combination of the above implementations for selective decarboxylation) for the second aerosolizable material to be located on a substrate which is located within the system such that, in use, aerosol from the first aerosolizable material contacts the substrate. In this regard, and in particular when the aerosol is formed via vaporization and subsequent condensation, the substrate will generally be exposed to an aerosol which is at a temperature which is significantly above ambient, such as greater than 50° C., greater than 60° C., greater than 70° C., greater than 80° C., greater than 90° C., or greater than 100° C. The temperature of the aerosol from the first aerosolizable material allows for the decarboxylation of the carboxylated active present on the substrate. The thus decarboxylated active can then be entrained in the aerosol and subsequently inhaled by the user.

In one example, it is possible for the user to vary the conditions of interaction between the aerosol from the first aerosolizable material and the substrate. For example, where the aerosol from the first aerosolizable material is generated via a heater, the relative distance between the substrate and the heater can be varied such that the closer the substrate is to the heater the higher the temperature of aerosol it is exposed to. It will also be appreciated that the substrate may experience radiative heat directly from the heater rather than merely through the aerosol and so moving the substrate closer to the heater will also increase the extent of radiative heating.

It is also possible for the extent to which the aerosol interacts with the substrate to be varied. In one instance, substantially all of the aerosol derived from the first aerosolizable material passes through the substrate. In other instances, a portion of, or indeed all of, the aerosol derived from the first aerosolizable material is able to by-pass the substrate. Accordingly, the user is able to control the extent to which the substrate is exposed to the aerosol and the heater.

In another aspect, the aerosol generating device comprises a power source, such as an electrical power source, a controller and at least one aerosol generator arranged to aerosolize the aerosolizable material to form an inhalable aerosol, wherein the controller is configured to facilitate delivery of power to the aerosol generator at more than one power level. The aerosol generator may be a heater. For the avoidance of doubt, the use of this alternative approach for the selective decarboxylation of the carboxylated active can be combined with the other approaches described herein for the selective decarboxylation of the carboxylated active.

During use, and due to the presence of the controller with variable power delivery to the aerosol generator (e.g. the heater), it is possible for the user to operate the system so as to control the extent of in situ conversion of the carboxylated form to the decarboxylated form. Since the rate of in situ conversion for some cannabinoids will generally be dependent on temperature (see Cannabis and Cannabinoid Research. Volume 1.1, 2016, Decarboxylation Study of Acidic Cannabinoids: A Novel Approach Using Ultra-High-Performance Supercritical Fluid Chromatography/Photodiode Array-Mass Spectrometry) providing a higher power to an aerosol generator, e.g. a heater, will generally result in a higher localized temperature at the heater meaning that conversion from the carboxylated form to the decarboxylated form will generally be greater. As such, the user is able to control the system so as to provide an aerosol with varying amounts of decarboxylated active. For example, where the carboxylated active is CBDA, the user is able to control the system so as to provide an aerosol with varying amounts of CBD.

The controller may be configured to facilitate delivery of power to the aerosol generator (e.g. heater) at more than one power level in a number of ways. For example, the controller may be configured to deliver power to the heater according to a “normal” power profile, and an “elevated” power profile. A normal power profile typically corresponds to a power profile delivered to the heater of the device when a user is not seeking a particularly elevated content of decarboxylated active in the subsequent aerosol. An elevated power profile corresponds to a power which is at or above a particular threshold power, that threshold power being a power that is greater than a power applied during the normal power profile.

Indeed, the aerosolizable material may already comprise some decarboxylated active (e.g. cannabinoid), and thus the normal power profile results in an aerosol with a “baseline” amount of decarboxylated active (e.g. cannabinoid). The precise power that is to be delivered during such a normal power profile is system specific. Moreover, the precise power to be delivered during the elevated power profile may depend on the concentration of carboxylated active in the aerosolizable material, as well as the transfer efficiency of energy from the aerosol generator to the aerosolizable material. For any particular system and aerosolizable material, the elevated power profile can be set to achieve a particular level (or minimum level) of decarboxylation above the “baseline” resulting from the normal power profile. However, the elevated power profile would typically correspond to power at or above a threshold power where that threshold power represents an increase in power relative to a power applied during the normal power profile of greater than 5%, greater than 10%, greater than 15%, greater than 20%, greater than 25%, greater than 30%, greater than 35%, greater than 40%, greater than 45%, greater than 50%, greater than 60%, greater than 70%, greater than 80%, greater than 90%, greater than 100%, greater than 110%, greater than 120%, greater than 130%, greater than 140%, greater than 150%, greater than 160%, greater than 170%, greater than 180%, greater than 190%, or greater than 200%. For example, if the normal power profile for a particular system is 3 W, the elevated power profile could be greater than 4.5 W (which would represent an increase in power of greater than 50%). Another example would be if the normal power profile for a particular system is 5 W, the elevated power profile could be greater than 12 W (which would represent an increase in power of greater than 100%).

It will be appreciated that the device may generally be actuated to provide a normal power profile in a number of ways. For example, the device could be puff actuated (in the sense that a sensor detects the presence of an inhalation, via for example a change in pressure or airflow), or the device could be manually actuated by a button, switch, touchpad or the like.

In order for the controller to deliver power to the heater according to the elevated power profile, the controller may be configured to respond to a particular input indicative of the desire for such an elevated power profile. For example, the device may comprise a button, switch, touchpad or the like, and the controller may detect actuation by the user of said button, switch, touchpad or the like. The button, switch, touchpad or the like may be dedicated to the provision of the “elevated” power profile. Alternatively, the controller may be programmed to detect a specific profile of actuation of an existing button, switch, touchpad or the like and recognize that specific profile of actuation as instruction to deliver the elevated power profile. The specific profile of actuation in relation to the elevated power profile is typically different to the profile of actuation to deliver the normal power profile. This allows for the controller to distinguish between actuations which are intended to result in a normal or elevated power profile. For example, the specific profile of actuation for the “elevated” power profile could entail a specific number of actuations or “taps” of the button, switch, touchpad or the like, such as a double or triple tap in short succession, e.g. within 1 or 2 seconds of each other.

Alternatively, the specific profile of actuation in relation to the elevated power profile corresponds to a particular actuation pressure, e.g. actuating over a certain pressure threshold. It is also possible for the applied elevated power to be proportional to the pressure applied. This allows correlation between the extent of actuation and the power delivered to the aerosol generator (e.g. heater), which allows for an intuitive application of the elevated power profile.

The controller can be configured to apply the elevated power profile for the current inhalation, for the next inhalation, and/or for all subsequent inhalations.

The controller may be configured to restrict the ability to apply the elevated power profile according to a predetermined schedule. For example, the elevated power profile may only be “accessible” to the user after a certain time of day, or based on a certain previous pattern of use of the system. In such circumstances, the controller would be configured to ignore the specific profile of actuation in relation to the “elevated” power profile. The predetermined schedule can be set by the user via the device, or via a remote device which communicates with the device of the system as described elsewhere.

Alternatively, the controller may be configured to automatically apply the elevated power profile according to a predetermined schedule. For example, the elevated power profile may be activated after a certain time of day, or based on a certain previous pattern of use of the system. In such circumstances, the controller would apply the elevated power profile for all instances of aerosol generation according to the schedule.

The controller might be configured to store a range of power level settings for the elevated power profile, and the user can access and select the desired higher setting for a particular inhalation. Such a selection could either be done via a button, switch, touchpad or the like on the device. The power level could be adjusted via another device which is remote to the device of the delivery system and yet can communicate with it, e.g. the power levels could be adjusted via an app running on a smartphone or tablet and the smartphone or tablet will communicate with the device of the delivery system to update the power settings.

The aerosol generator is typically a heater. Where the aerosol generator is a heater, the temperature at the heater will generally be influenced by the power provided to the heater, such that a higher power will promote a higher heater temperature. The skilled person will be aware that other factors might influence the precise temperature of the heater, such as airflow past the heater, or the rate at which aerosolizable material can be replaced at a location in proximity to the heater. In some embodiments, it is envisaged that the system allows for the variation in airflow past the aerosol generator (e.g. heater) and/or allows for the variation in the rate of delivery of aerosolizable material to the aerosol generator (e.g. heater). One way of varying the airflow would be to modify the total area of one or more air inlets of the system. This could be done via a shutter or the like. One way of varying the rate of delivery of aerosolizable material to the aerosol generator would be to use a pump with different flow rates.

An alternative way the system may provide for the selective decarboxylation of the carboxylated active is for the system to be configured to deliver puffs for varying lengths of time. It has been found that longer puffs can lead to greater relative percentages of the decarboxylated active in the aerosol. By configuring the system to deliver a puff of a certain length, it is possible to selectively influence the extent of decarboxylation. Accordingly, the device may be pre-configured to deliver a certain puff length (the user being able to change the pre-configured puff length so as to increase the decarboxylation) and/or the device may configured to prompt the user to puff for a certain length of time in order to influence the extent of decarboxylation).

In a further aspect, there is provided an article comprising an aerosolizable material, wherein the aerosolizable material comprises at least one present carboxylated active.

The article may comprise at least a first reservoir for containing the aerosolizable material. The article may comprise first and second reservoirs for containing first and second aerosolizable materials as described herein.

The aerosolizable material(s) generally comprise one or more carboxylated actives, a carrier constituent and optionally one or more flavors. Generally, the aerosolizable material(s) takes the form of a liquid. It will be appreciated that this liquid can be held freely within a reservoir of the device, or might be retained on a carrier.

As described above, the aerosolizable material(s) contain at least one carboxylated active. In some embodiments, the substance to be delivered comprises an active substance.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, Cannabis or another botanical.

In one embodiment the active substance is a legally permissible recreational drug.

In some embodiments, the active substance comprises nicotine. In some embodiments, the active substance comprises caffeine, melatonin or vitamin B12. In some embodiments, the active comprises a cannabinoid. Preferably, the carboxylated active is a carboxylated cannabinoid.

Cannabinoids are a class of natural or synthetic chemical compounds which act on cannabinoid receptors (i.e., CB1 and CB2) in cells that repress neurotransmitter release in the brain. Cannabinoids are cyclic molecules exhibiting particular properties such as the ability to easily cross the blood-brain barrier. Cannabinoids may be naturally occurring (Phytocannabinoids) from plants such as Cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabigerolic acid (CBGA), cannabichromene (CBC), cannabichromenic acid (CBCA), cannabidiol (CBD), cannabidolic acid (CBDA), tetrahydrocannabinol (THC) including its isomers Δ^{6a,10a}-tetrahydrocannabinol (Δ^{6a,10a}-THC), Δ^{6a(7)}-tetrahydrocannabinol (Δ^{6a(7)}-THC), Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), Δ^{9,11}-tetrahydrocannabinol (Δ^{9,11}-THC), tetrahydrocannabinolic acid (THCA), cannabinol (CBN), cannabinolic acid (CBNA), and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), and cannabigerol monomethyl ether (CBGM).

Naturally derived cannabinoids are generally present in their carboxylated form. In this regard, cannabidiol (CBD) and cannabidolic acid (CBDA) are the respective decarboxylated and carboxylated forms.

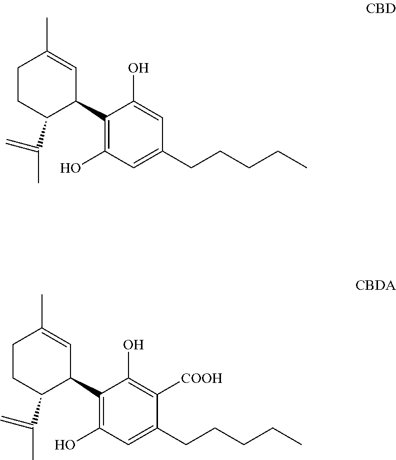

The carboxylated cannabinoid referred to herein may be the carboxylated form of any of the decarboxylated cannabinoids mentioned above. Preferably, the carboxylated cannabinoid is cannabigerolic acid (CBGA), cannabichromenic acid (CBCA), cannabinolic acid (CBNA), tetrahydrocannabinolic acid (THCA), cannabidolic acid (CBDA) and combinations thereof. Preferably, the decarboxylated cannabinoid is cannabigerol (CBG), cannabichromene (CBC), cannabinol (CBN), tetrahydrocannabinol (THC), cannabidiol (CBD) and combinations thereof.

The aerosolizable material(s) may also comprise one or more other actives which are in the decarboxylated form. Preferably, the one or more other actives are cannabinoids. Preferably, the one or more other cannabinoid is the decarboxylated form of the carboxylated cannabinoid. Thus, where the aerosolizable material comprises CBDA it may also comprise CBD. Preferably, the aerosolizable material contains CBDA and CBD. However, it may be that the other active is a decarboxylated cannabinoid that does not result from decarboxylation of the carboxylated cannabinoid present in the aerosolizable formulation.

In some embodiments, the aerosolizable material, which comprises at least one carboxylated cannabinoid, may also comprise one or more of cannabigerol (CBG), cannabichromene (CBC), cannabidiol (CBD), tetrahydrocannabinol (THC), and cannabinol (CBN). In some embodiments, the aerosolizable material comprises cannabidolic acid (CBDA), and at least one decarboxylated cannabinoid selected from the group consisting of cannabigerol (CBG), cannabichromene (CBC), cannabidiol (CBD), tetrahydrocannabinol (THC), and cannabinol (CBN). In one embodiment, the aerosolizable material comprises cannabidolic acid (CBDA) and tetrahydrocannabinol (THC).

The respective molar ratio of the carboxylated/decarboxylated forms of the active in the aerosolizable material may be varied. For example, the molar ratio of carboxylated active to its corresponding decarboxylated active may be from 99:1 to 1:99. Preferably, the actives are cannabinoids and the molar ratio of carboxylated cannabinoid to its corresponding decarboxylated cannabinoid may be from 99:1 to 1:99. Particular ratios in this regard may be 10:1, 9:1, 8:1, 7:1, 6:1, 5:1, 4:1, 3:1, 2:1, 1:1, 1:2, 1:3, 1:4, or 1:5. Preferably, the carboxylated form is present in a molar excess relative to the decarboxylated form.

The cannabinoids may be synthetic or natural in origin. In one embodiment, the cannabinoids are present in the form of an isolate. An isolate is an extract from a plant, such as Cannabis, where the active material of interest (in this case the cannabinoid, such as CBDA) is present in a high degree of purity, for example greater than 95%, greater than 96%, greater than 97%, greater than 98%, or around 99% purity.

The cannabinoids (whether carboxylated or decarboxylated) may be present in the aerosolizable material based on a mg/ml basis of the aerosolizable material. Reference below to “the” cannabinoid relates individually to each cannabinoid present in the aerosolizable material, whether that be carboxylated or not. The below amounts are provided in the context of cannabinoids, but each of the below ranges can be applied equally to other actives (carboxylated or decarboxylated) referred to herein.

In one embodiment, the cannabinoid is present in an amount of from about 5 mg/ml up to about 200 mg/ml. In one embodiment, the cannabinoid is present in an amount of from about 5 mg/ml up to about 150 mg/ml. In one embodiment, the cannabinoid is present in an amount of from about 5 mg/ml up to about 90 mg/ml. In one embodiment, the cannabinoid is present in an amount of from about 5 mg/ml up to about 80 mg/ml. In one embodiment, the cannabinoid is present in an amount of from about 5 mg/ml up to about 70 mg/ml. In one embodiment, the cannabinoid is present in an amount of from about 5 mg/ml up to about 60 mg/ml. In one embodiment, the cannabinoid is present in an amount of from about 5 mg/ml up to about 50 mg/ml. In one embodiment, the cannabinoid is present in an amount of from about 5 mg/ml up to about 40 mg/ml. In one embodiment, the cannabinoid is present in an amount of from about 5 mg/ml up to about 30 mg/ml. In one embodiment, the cannabinoid is present in an amount of from about 5 mg/ml up to about 20 mg/ml. In one embodiment, the cannabinoid is present in an amount of from about 5 mg/ml up to about 10 mg/ml.

In one embodiment, the cannabinoid is present in an amount of about 5 mg/ml or more. In one embodiment, the cannabinoid is present in an amount of about 10 mg/ml or more. In one embodiment, the cannabinoid is present in an amount of about 15 mg/ml or more. In one embodiment, the cannabinoid is present in an amount of about 20 mg/ml or more. In one embodiment, the cannabinoid is present in an amount of about 25 mg/ml or more. In one embodiment, the cannabinoid is present in an amount of about 30 mg/ml or more. In one embodiment, the cannabinoid is present in an amount of about 35 mg/ml or more. In one embodiment, the cannabinoid is present in an amount of about 40 mg/ml or more. In one embodiment, the cannabinoid is present in an amount of about 45 mg/ml or more. In one embodiment, the cannabinoid is present in an amount of about 50 mg/ml or more. In one embodiment, the cannabinoid is present in an amount of about 55 mg/ml or more. In one embodiment, the cannabinoid is present in an amount of about 60 mg/ml or more. In one embodiment, the cannabinoid is present in an amount of about 65 mg/ml or more.

The total amount of all actives present in the or each aerosolizable material be may be 200 mg/ml.

The total amount of all cannabinoids present in the or each aerosolizable material be may be 200 mg/ml.

The carrier constituent comprises one or more constituents capable of forming an aerosol, particularly when evaporated and allowed to condense. In some embodiments, the carrier constituent may comprise one or more of glycerol, propylene glycol, triethylene glycol, tetraethylene glycol, 1,3-butylene glycol, erythritol, meso-Erythritol, ethyl vanillate, ethyl laurate, a diethyl suberate, triethyl citrate, triethylene glycol diacetate, triacetin, a diacetin mixture, benzyl benzoate, benzyl phenyl acetate, tributyrin, lauryl acetate, lauric acid, myristic acid, and propylene carbonate. Preferably, the carrier constituent comprises propylene glycol and/or glycerol.

In one embodiment, propylene glycol is present in an amount of from 10% w/w to 95% w/w based on the total weight of the aerosolizable material. In one embodiment, propylene glycol is present in an amount of from 20% w/w to 95% w/w based on the total weight of the material. In one embodiment, propylene glycol is present in an amount of from 30% w/w to 95% w/w based on the total weight of the material. In one embodiment, propylene glycol is present in an amount of from 40% w/w to 95% w/w based on the total weight of the material.

In one embodiment, propylene glycol is present in an amount of from 50% w/w to 90% w/w based on the total weight of the material. In one embodiment, propylene glycol is present in an amount of from 50% w/w to 85% w/w based on the total weight of the material. In one embodiment, propylene glycol is present in an amount of from 50% w/w to 80% w/w based on the total weight of the material. In one embodiment, propylene glycol is present in an amount of from 50% w/w to 75% w/w based on the total weight of the material. In one embodiment, propylene glycol is present in an amount of from 50% w/w to 60% w/w based on the total weight of the material. In one embodiment, propylene glycol is present in an amount of from 50% w/w to 65% w/w based on the total weight of the material. In one embodiment, propylene glycol is present in an amount of from 50% w/w to 60% w/w based on the total weight of the material.

In one embodiment, propylene glycol is present in an amount of from 55% w/w to 90% w/w based on the total weight of the material. In one embodiment, propylene glycol is present in an amount of from 60% w/w to 90% w/w based on the total weight of the material. In one embodiment, propylene glycol is present in an amount of from 65% w/w to 90% w/w based on the total weight of the material. In one embodiment, propylene glycol is present in an amount of from 70% w/w to 90% w/w based on the total weight of the material. In one embodiment, propylene glycol is present in an amount of from 75% w/w to 90% w/w based on the total weight of the material. In one embodiment, propylene glycol is present in an amount of from 80% w/w to 90% w/w based on the total weight of the material. In one embodiment, propylene glycol is present in an amount of from 85% w/w to 90% w/w based on the total weight of the material.

In one embodiment, propylene glycol is present in an amount of at least 10% w/w based on the total weight of the material. In one embodiment, propylene glycol is present in an amount of at least 20% w/w based on the total weight of the material. In one embodiment, propylene glycol is present in an amount of at least 30% w/w based on the total weight of the material. In one embodiment, propylene glycol is present in an amount of at least 40% w/w based on the total weight of the material. In one embodiment, propylene glycol is present in an amount of at least 50% w/w based on the total weight of the material. In one embodiment, propylene glycol is present in an amount of at least 55% w/w based on the total weight of the material. In one embodiment, propylene glycol is present in an amount of at least 60% w/w based on the total weight of the material. In one embodiment, propylene glycol is present in an amount of at least 65% w/w based on the total weight of the material. In one embodiment, propylene glycol is present in an amount of at least 70% w/w based on the total weight of the material. In one embodiment, propylene glycol is present in an amount of at least 75% w/w based on the total weight of the material. In one embodiment, propylene glycol is present in an amount of at least 80% w/w based on the total weight of the material. In one embodiment, propylene glycol is present in an amount of at least 85% w/w based on the total weight of the material. In one embodiment, propylene glycol is present in an amount of at least 90% w/w based on the total weight of the material.

In one embodiment, the carrier constituent comprises glycerol. In one embodiment, glycerol is present in an amount of from 10% w/w to 95% w/w based on the total weight of the material. In one embodiment, glycerol is present in an amount of from 20% w/w to 95% w/w based on the total weight of the material. In one embodiment, glycerol is present in an amount of from 30% w/w to 95% w/w based on the total weight of the material. In one embodiment, glycerol is present in an amount of from 40% w/w to 95% w/w based on the total weight of the material. In one embodiment, glycerol is present in an amount of from 50% w/w to 95% w/w based on the total weight of the material.

In one embodiment, glycerol is present in an amount of from 50% w/w to 90% w/w based on the total weight of the material. In one embodiment, glycerol is present in an amount of from 50% w/w to 85% w/w based on the total weight of the material. In one embodiment, glycerol is present in an amount of from 50% w/w to 80% w/w based on the total weight of the material. In one embodiment, glycerol is present in an amount of from 50% w/w to 75% w/w based on the total weight of the material. In one embodiment, glycerol is present in an amount of from 50% w/w to 60% w/w based on the total weight of the material. In one embodiment, glycerol is present in an amount of from 50% w/w to 65% w/w based on the total weight of the material. In one embodiment, glycerol is present in an amount of from 50% w/w to 60% w/w based on the total weight of the material.

In one embodiment, glycerol is present in an amount of from 55% w/w to 90% w/w based on the total weight of the material. In one embodiment, glycerol is present in an amount of from 60% w/w to 90% w/w based on the total weight of the material. In one embodiment, glycerol is present in an amount of from 65% w/w to 90% w/w based on the total weight of the material. In one embodiment, glycerol is present in an amount of from 70% w/w to 90% w/w based on the total weight of the material. In one embodiment, glycerol is present in an amount of from 75% w/w to 90% w/w based on the total weight of the material. In one embodiment, glycerol is present in an amount of from 80% w/w to 90% w/w based on the total weight of the material. In one embodiment, glycerol is present in an amount of from 85% w/w to 90% w/w based on the total weight of the material.

In one embodiment, glycerol is present in an amount of at least 10% w/w based on the total weight of the material. In one embodiment, glycerol is present in an amount of at least 20% w/w based on the total weight of the material. In one embodiment, glycerol is present in an amount of at least 30% w/w based on the total weight of the material. In one embodiment, glycerol is present in an amount of at least 40% w/w based on the total weight of the material. In one embodiment, glycerol is present in an amount of at least 50% w/w based on the total weight of the material. In one embodiment, glycerol is present in an amount of at least 50% w/w based on the total weight of the material. In one embodiment, glycerol is present in an amount of at least 55% w/w based on the total weight of the material. In one embodiment, glycerol is present in an amount of at least 60% w/w based on the total weight of the material. In one embodiment, glycerol is present in an amount of at least 65% w/w based on the total weight of the material. In one embodiment, glycerol is present in an amount of at least 70% w/w based on the total weight of the material. In one embodiment, glycerol is present in an amount of at least 75% w/w based on the total weight of the material. In one embodiment, glycerol is present in an amount of at least 80% w/w based on the total weight of the material. In one embodiment, glycerol is present in an amount of at least 85% w/w based on the total weight of the material. In one embodiment, glycerol is present in an amount of at least 90% w/w based on the total weight of the material.

In one embodiment, both glycerol and propylene glycol are present as carrier constituents. In one embodiment, glycerol and propylene glycol are present in the aerosolizable material in the following amounts:
60 to 90% w/w propylene glycol; and
40 to 10% w/w glycerol,
based on the total weight of glycerol and propylene glycol present in the material.
In one embodiment, glycerol and propylene glycol are present in the aerosolizable material in the following amounts:
70 to 80% w/w propylene glycol; and
30 to 20% w/w glycerol.
based on the total weight of glycerol and propylene glycol present in the material.

In one embodiment, the aerosolizable material comprises about 70% w/w propylene glycol and about 30% glycerol.

In one embodiment, the aerosolizable material is a liquid at about 25° C.

The aerosolizable material may comprise one or more further constituents. In particular, one or more further constituents may be selected from one or more physiologically and/or olfactory active constituents, and/or one or more functional constituents.

In some embodiments, the active constituent is an olfactory active constituent and may be selected from a “flavor” and/or “flavorant” which, where local regulations permit, may be used to create a desired taste, aroma or sensation in a product for adult consumers. In some instances such constituents may be referred to as flavors, flavorants, cooling agents, heating agents, or sweetening agents, and may include one or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

The flavor may be added to the aerosolizable material as part of a so-called “flavor block”, where one or more flavors are blended together and then added to the aerosolizable material.

In some embodiments, the olfactory active constituent comprises a terpene. In some embodiments, the terpene is a terpene derivable from a phytocannabinoid producing plant, such as a plant from the strain of the Cannabis sativa species, such as hemp. In some embodiments, the aerosolizable material comprises a cannabinoid isolate in combination with a terpene derivable from a phytocannabinoid producing plant.

Suitable terpenes in this regard include so-called “C10” terpenes, which are those terpenes comprising 10 carbon atoms. Further, suitable terpenes in this regard also include so-called “C15” terpenes, which are those terpenes comprising 15 carbon atoms. In some embodiments, the aerosolizable material comprises more than one terpene. For example, the aerosolizable material may comprise one, two, three, four, five, six, seven, eight, nine, ten or more terpenes as defined herein.

In some embodiments, the aerosolizable material comprises a combination of terpenes. In some embodiments, the combination of terpenes may comprise a combination of at least geraniol and linalool. In some embodiments, the combination of terpenes may comprise a combination of at least eucalyptol and menthone. In some embodiments, the combination of terpenes may comprise a combination of at least eucalyptol, carvone, piperitone and menthone. In some embodiments, the combination of terpenes may comprise a combination of at least eucalyptol, carvone, beta-bourbonene, germacrene, piperitone, iso-menthone and menthone.

In one embodiment, the terpene(s) are present in a flavor block. This means that the terpenes are blended with one or more other flavors (optionally with an appropriate solvent, for example propylene glycol) and then the flavor block is added during the manufacture of the aerosolizable material. In some embodiments, the total amount of the flavor block present in the aerosolizable material is up to about 10 w/w %. In some embodiments, the total amount of the flavor block present in the aerosolizable material is up to about 9 w/w %. In some embodiments, the total amount of the flavor block present in the aerosolizable material is up to about 8 w/w %. In some embodiments, the total amount of the flavor block present in the aerosolizable material is up to about 7 w/w %. In some embodiments, the total amount of the flavor block present in the aerosolizable material is up to about 6 w/w %. In some embodiments, the total amount of the flavor block present in the aerosolizable material is up to about 5 w/w %.

In one embodiment, the total amount of terpene present in the aerosolizable material is up to about 10 mg/ml. In one embodiment, the total amount of terpene present in the aerosolizable material is up to about 9 mg/ml. In one embodiment, the total amount of terpene present in the aerosolizable material is up to about 8 mg/ml. In one embodiment, the total amount of terpene present in the aerosolizable material is up to about 7 mg/ml. In one embodiment, the total amount of terpene present in the aerosolizable material is up to about 6 mg/ml. In one embodiment, the total amount of terpene present in the aerosolizable material is up to about 5 mg/ml. In one embodiment, the total amount of terpene present in the aerosolizable material is up to about 4 mg/ml. In one embodiment, the total amount of terpene present in the aerosolizable material is up to about 3 mg/ml. In one embodiment, the total amount of terpene present in the aerosolizable material is up to about 2 mg/ml. In one embodiment, the total amount of terpene present in the aerosolizable material is up to about 1 mg/ml.

In one embodiment, the total amount of terpene present in the aerosolizable material is from about 0.1 mg/ml up to about 10 mg/ml. In one embodiment, the total amount of terpene present in the aerosolizable material is from about 0.2 mg/ml up to about 10 mg/ml. In one embodiment, the total amount of terpene present in the aerosolizable material is from about 0.3 mg/ml up to about 10 mg/ml. In one embodiment, the total amount of terpene present in the aerosolizable material is from about 0.4 mg/ml up to about 10 mg/ml. In one embodiment, the total amount of terpene present in the aerosolizable material is from about 0.5 mg/ml up to about 10 mg/ml. In one embodiment, the total amount of terpene present in the aerosolizable material is from about 1.0 mg/ml up to about 10 mg/ml. In one embodiment, the total amount of terpene present in the aerosolizable material is from about 2.0 mg/ml up to about 10 mg/ml. In one embodiment, the total amount of terpene present in the aerosolizable material is from about 3.0 mg/ml up to about 10 mg/ml. In one embodiment, the total amount of terpene present in the aerosolizable material is from about 4.0 mg/ml up to about 10 mg/ml. In one embodiment, the total amount of terpene present in the aerosolizable material is from about 5.0 mg/ml up to about 10 mg/ml.

In one embodiment, the total amount of terpene present in the aerosolizable material is from about 0.1 mg/ml up to about 9.0 mg/ml. In one embodiment, the total amount of terpene present in the aerosolizable material is from about 0.1 mg/ml up to about 8.0 mg/ml. In one embodiment, the total amount of terpene present in the aerosolizable material is from about 0.1 mg/ml up to about 7.0 mg/ml. In one embodiment, the total amount of terpene present in the aerosolizable material is from about 0.1 mg/ml up to about 6.0 mg/ml. In one embodiment, the total amount of terpene present in the aerosolizable material is from about 0.1 mg/ml up to about 5.0 mg/ml. In one embodiment, the total amount of terpene present in the aerosolizable material is from about 0.1 mg/ml up to about 1 mg/ml. In one embodiment, the total amount of terpene present in the aerosolizable material is from about 0.1 mg/ml up to about 0.9 mg/ml. In one embodiment, the total amount of terpene present in the aerosolizable material is from about 0.1 mg/ml up to about 0.8 mg/ml. In one embodiment, the total amount of terpene present in the aerosolizable material is from about 0.1 mg/ml up to about 0.7 mg/ml. In one embodiment, the total amount of terpene present in the aerosolizable material is from about 0.1 mg/ml up to about 0.6 mg/ml. In one embodiment, the total amount of terpene present in the aerosolizable material is from about 0.1 mg/ml up to about 0.5 mg/ml.

For the avoidance of doubt, combinations of the above end points are explicitly envisaged by the present disclosure. This applies to any of the ranges disclosed herein.

The one or more other functional constituents may comprise one or more of pH regulators, colouring agents, preservatives, binders, fillers, stabilizers, and/or antioxidants. In particular, the pH regulator may include one or more acids selected from organic or inorganic acids. An example of an inorganic acid is phosphoric acid. The organic acid may include a carboxylic acid. The carboxylic acid may be any suitable carboxylic acid. In one embodiment the acid is a mono-carboxylic acid. In one embodiment the acid may be selected from the group consisting of acetic acid, lactic acid, formic acid, citric acid, benzoic acid, pyruvic acid, levulinic acid, succinic acid, tartaric acid, oleic acid, sorbic acid, propionic acid, phenylacetic acid, and mixtures thereof.

The aerosolizable material may also comprise water. For example, water could be present in amounts up to 10% w/w based on the total weight of the aerosolizable material. In one embodiment, water is present in the aerosolizable material in an amount of up to about 5% w/w. In one embodiment, water is present in the aerosolizable material in an amount of up to about 3% w/w. In one embodiment, water is present in the aerosolizable material in an amount of about 1% w/w. In one embodiment, water is present in the aerosolizable material in an amount of about 1% w/w. In one embodiment, water is present in the aerosolizable material in an amount of about 2% w/w. In one embodiment, water is present in the aerosolizable material in an amount of about 3% w/w. In one embodiment, water is present in the aerosolizable material in an amount of about 4% w/w. In one embodiment, water is present in the aerosolizable material in an amount of about 5% w/w.

It is suggested that the presence of a degree of water may promote the decarboxylation process.

In one embodiment, the aerosolizable material may comprise
1 to 10% w/w carboxylated active;
60 to 80% w/w propylene glycol;
20 to 30% w/w glycerol; and
0 to 10% w/w water
based on the total weight of the material.

In one embodiment, the aerosolizable material may comprise
about 5% w/w carboxylated active;
about 70% w/w propylene glycol;
about 25% w/w glycerol; and
0% w/w water
based on the total weight of the material.

In one embodiment, the aerosolizable material may comprise
about 5% w/w carboxylated active;
about 70% w/w propylene glycol;
about 22% w/w glycerol; and
3% w/w water
based on the total weight of the material.

It may be advantageous to use water containing formulations with devices configured to deliver a certain, e.g. lower, power to the aerosolizable formulation. Therefore, in one embodiment, there is provided a delivery system comprising a powered aerosol generating device and an aerosolizable material, wherein the aerosolizable material comprises at least one carboxylated active and water, and the powered aerosol generating device is configured to deliver less than 12 W per puff to the aerosolizable material. In one embodiment, the powered aerosol generating device is configured to deliver less than 11 W, 10 W, 9 W, 8 W, 7 W, 6 W, or 5 W to the aerosolizable material.

There is also provided an aerosolizable material as defined herein. In particular any of the constituents and their respective amounts described herein may be used to characterize the aerosolizable material.

### BRIEF DESCRIPTION OF FIGURES

FIG. 1 is a schematic diagram (not to scale) of an example delivery system, such as an e-cigarette according to the present disclosure;

FIG. 2 provides a schematic view of part of an airflow channel through an aerosol delivery system as described herein;

FIG. 2A provides a schematic view of a removable segment according to the present disclosure;

FIG. 3 provides a schematic view of part of an airflow channel through an aerosol delivery system as described herein;

FIG. 4 provides a schematic view of an aerosol provision system where a first reservoir and a second reservoir are located

### DETAILED DESCRIPTION

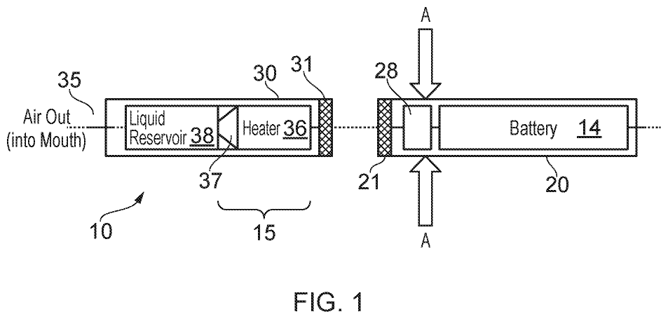

FIG. 1 is a highly schematic diagram (not to scale) of an example delivery system, such as an e-cigarette 10, to which embodiments are applicable. The e-cigarette has a generally cylindrical shape, extending along a longitudinal axis indicated by a dashed line (although aspects of the invention are applicable to e-cigarettes configured in other shapes and arrangements), and comprises two main components, namely an aerosol provision device 20 and an article 30.

The article 30 includes a store or reservoir for aerosolizable material (source liquid) 38 from which an aerosol is to be generated. The article 30 further comprises an aerosol generating component (such as heating element or heater) 36 for heating the aerosolizable material to generate the aerosol. A transport element or wicking element or wick 37 is provided to deliver aerosolizable material from the store 38 to the heating element 36. A part or parts of the wick 37 are in fluid communication with aerosolizable material in the store 38 and by a wicking or capillary action aerosolizable material is drawn along or through the wick 37 to a part or parts of the wick 37 which are in contact with the heater 36. The skilled person will appreciate that other modes of transporting liquid to a heater can be used, such as pumping, dripping or the like.

Vaporization of the aerosolizable material occurs at the interface between the wick 37 and the heater 36 by the provision of heat energy to the aerosolizable material to cause evaporation, thus generating the aerosol. The wick 37 and the heater 36 may be collectively referred to as a vaporizer or an atomiser 15.

Typically a single wick will be present, but it is envisaged that more than one wick could be present, for example, two, three, four or five wicks.

As described above, the wick may be formed a sintered material. The sintered material may comprise sintered ceramic, sintered metal fibers/powders, or a combination of the two. The (or at least one of/all of the) sintered wick(s) may have deposited thereon/embedded therein an electrically resistive heater. Such a heater may be formed from heat conducting alloys such as NiCr alloys. Alternatively, the sintered material may have such electrical properties such that when a current is passed there through, it is heated. Thus, the aerosol generating component and the wick may be considered to be integrated. In some embodiments, the aerosol generating component and the wick are formed from the same material and form a single component.

In some embodiments, the wick is formed from a sintered metal material and is generally in the form of a planar sheet. Thus, the wick element may have a substantially thin flat shape. For example it may be considered as a sheet, layer, film, substrate or the like. By this it is meant that a thickness of the wick is less or very much less than at least one of the length and the width of the wick. Thus, the wick thickness (its smallest dimension) is less or very much less than the longest dimension.

The wick may be made of a homogenous, granular, fibrous or flocculent sintered metal(s) so as to form said capillary structure. Wick elements can be made from a conductive material which is a nonwoven sintered porous web structure comprising metal fibers, such as fibers of stainless steel. For example, the stainless steel may be AISI (American Iron and Steel Institute) 316L (corresponding to European standard 1.4404). The material's weight may be in the range of 100-300 g/m².

Where the wick is generally planar, the thickness of the wick may be in the range of 75-250 μm. A typical fiber diameter may be about 12 μm, and a typical mean pore size (size of the voids between the fibers) may be about 32 μm. An example of a material of this type is Bekipor (RTM) ST porous metal fiber media manufactured by NV Bekaert SA, Belgium, being a range of porous nonwoven fiber matrix materials made by sintering stainless steel fibers.

Note also that while the material is described as planar, this refers to the relative dimensions of the sheet material and the wick (a thickness many times smaller than the length and/or width) but does not necessarily indicate flatness, in particular of the final wick made from the material. A wick may be flat but might alternatively be formed from sheet material into a non-flat shape such as curved, rippled, corrugated, ridged, formed into a tube or otherwise made concave and/or convex.

The wick element may have various properties. It is formed from a porous material to enable the required wicking or capillary effect for drawing source liquid through it from an store for aerosolizable material (where the wick meets the aerosolizable material at a store contact site) to the vaporization interface. Porosity is typically provided by a plurality of interconnected or partially interconnected pores (holes or interstices) throughout the material, and open to the outer surface of the material. Any level of porosity may be employed depending on the material, the size of the pores and the required rate of wicking. For example a porosity of between 30% and 85% might be selected, such as between 40% and 70%, between 50% and 80%, between 35% and 75% or between 40% and 75%. This might be an average porosity value for the whole wick element, since porosity may or may not be uniform across the wick. For example, pore size at the store contact site might be different from pore size nearer to the heater.

It is useful for the wick to have sufficient rigidity to support itself in a required within the article. For example, it may be mounted at or near one or two edges and be required to maintain its position substantially without flexing, bending or sagging.

As an example, porous sintered ceramic is a useful material to use as the wick element. Any ceramic with appropriate porosity may be used. If porous ceramic is chosen as the porous wick material, this is available as a powder which can be formed into a solid by sintering (heating to cause coalescence, possibly under applied pressure). Sintering then solidifies the ceramic to create the porous wick.

The article 30 further includes a mouthpiece 35 having an opening through which a user may inhale the aerosol generated by the vaporizer 15. The aerosol for inhalation may be described as an aerosol stream or inhalable airstream.

The aerosol delivery device 20 includes a power source (a re-chargeable cell or battery 14, referred to herein after as a battery) to provide power for the e-cigarette 10, and a controller (printed circuit board (PCB)) 28 and/or other electronics for generally controlling the e-cigarette 10. The aerosol delivery device can therefore also be considered as a battery section, or a control unit or section.

During operation of the device, the controller will determine that a user has initiated a request for the generation of an aerosol. This could be done via a button on the device which sends a signal to the controller that the aerosol generator should be powered. Alternatively, a sensor located in or proximal to the airflow pathway could detect airflow through the airflow pathway and convey this detection to the controller. A sensor may also be present in addition to the presence of a button, as the sensor may be used to determine certain usage characteristics, such as airflow, timing of aerosol generation etc.

For example, in use, when the heater 36 receives power from the battery 14, as controlled by the circuit board 28 possibly in response to pressure changes detected by an air pressure sensor (not shown), the heater 36 vaporizes aerosolizable material delivered by the wick 37 to generate the aerosol, and this aerosol stream is then inhaled by a user through the opening in the mouthpiece 35. The aerosol is carried from the aerosol source to the mouthpiece 35 along an air channel (not shown in FIG. 1) that connects the aerosol source to the mouthpiece opening as a user inhales on the mouthpiece.

In this particular example, the device 20 and article 30 are detachable from one another by separation in a direction parallel to the longitudinal axis, as shown in FIG. 1, but are joined together when the system 10 is in use by cooperating engagement elements 21, 31 (for example, a screw, magnetic or bayonet fitting) to provide mechanical and electrical connectivity between the device 20 and the article 30, in particular connecting the heater 36 to the battery 14. The battery may be charged as is known to one skilled in the art.

As mentioned, a type of aerosol generating component, such as a heating element, that may be utilized in an atomising portion of an electronic cigarette (a part configured to generate vapor from a source liquid) combines the functions of heating and liquid delivery, by being both electrically conductive (resistive) and porous. Note here that reference to being electrically conductive (resistive) refers to components which have the capacity to generate heat in response to the flow of electrical current therein. Such flow could be imparted by via so-called resistive heating or induction heating. An example of a suitable material for this is an electrically conductive material such as a metal or metal alloy formed into a sheet-like form, i.e. a planar shape with a thickness many times smaller than its length or breadth. Examples in this regard may be a mesh, web, grill and the like. The mesh may be formed from metal wires or fibers which are woven together, or alternatively aggregated into a non-woven structure. For example, fibers may be aggregated by sintering, in which heat and/or pressure are applied to a collection of metal fibers to compact them into a single porous mass.

These structures can give appropriately sized voids and interstices between the metal fibers to provide a capillary force for wicking liquid. Thus, these structures can also be considered to be porous since they provide for the uptake and distribution of liquid. Moreover, due to the presence of voids and interstices between the metal fibers, it is possible for air to permeate through said structures. Also, the metal is electrically conductive and therefore suitable for resistive heating, whereby electrical current flowing through a material with electrical resistance generates heat. Structures of this type are not limited to metals, however; other conductive materials may be formed into fibers and made into mesh, grill or web structures. Examples include ceramic materials, which may or may not be doped with substances intended to tailor the physical properties of the mesh.

A planar sheet-like porous aerosol generating component of this kind can be arranged within an electronic cigarette such that it lies within the aerosol generating chamber forming part of an airflow channel. The aerosol generating component may be oriented within the chamber such that air flow though the chamber may flow in a surface direction, i.e. substantially parallel to the plane of the generally planar sheet-like aerosol generating component. An example of such a configuration can be found in WO2010/045670 and WO2010/045671, the contents of which are incorporated herein in their entirety by reference. Air can thence flow over the heating element, and gather vapor. Aerosol generation is thereby made very effective. In alternative examples, the aerosol generating component may be oriented within the chamber such that air flow though the chamber may flow in a direction which is substantially transverse to the surface direction, i.e. substantially orthogonally to the plane of the generally planar sheet-like aerosol generating component. An example of such a configuration can be found in WO2018/211252, the contents of which are incorporated herein in its entirety by reference.

The aerosol generating component may have any one of the following structures: a woven or weave structure, mesh structure, fabric structure, open-pored fiber structure, open-pored sintered structure, open-pored foam or open-pored deposition structure. Said structures are suitable in particular for providing an aerosol generating component with a high degree of porosity. A high degree of porosity may ensure that the heat produced by the aerosol generating component is predominately used for evaporating the liquid and high efficiency can be obtained. A porosity of greater than 50% may be envisaged with said structures. In one embodiment, the porosity of the aerosol generating component is 50% or greater, 60% or greater, 70% or greater. The open-pored fiber structure can consist, for example, of a non-woven fabric which can be arbitrarily compacted, and can additionally be sintered in order to improve the cohesion. The open-pored sintered structure can consist, for example, of a granular, fibrous or flocculent sintered composite produced by a film casting process. The open-pored deposition structure can be produced, for example, by a CVD process, PVD process or by flame spraying. Open-pored foams are in principle commercially available and are also obtainable in a thin, fine-pored design.

In one embodiment, the aerosol generating component has at least two layers, wherein the layers contain at least one of the following structures: a plate, foil, paper, mesh, woven structure, fabric, open-pored fiber structure, open-pored sintered structure, open-pored foam or open-pored deposition structure. For example, the aerosol generating component can be formed by an electric heating resistor consisting of a metal foil combined with a structure comprising a capillary structure. Where the aerosol generating component is considered to be formed from a single layer, such a layer may be formed from a metal wire fabric, or from a non-woven metal fiber fabric. Individual layers are advantageously but not necessarily connected to one another by a heat treatment, such as sintering or welding. For example, the aerosol generating component can be designed as a sintered composite consisting of a stainless steel foil and one or more layers of a stainless steel wire fabric (material, for example AISI 304 or AISI 316). Alternatively the aerosol generating component can be designed as a sintered composite consisting of at least two layers of a stainless steel wire fabric. The layers may be connected to one another by spot welding or resistance welding. Individual layers may also be connected to one another mechanically. For instance, a double-layer wire fabric could be produced just by folding a single layer. Instead of stainless steel, use may also be made, by way of example, of heating conductor alloys-in particular NiCr alloys and CrFeAl alloys (“Kanthal”) which have an even higher specific electric resistance than stainless steel. The material connection between the layers is obtained by the heat treatment, as a result of which the layers maintain contact with one another-even under adverse conditions, for example during heating by the aerosol generating component and resultantly induced thermal expansions. Alternatively, the aerosol generating component may be formed from sintering a plurality of individual fibers together. This, the aerosol generating component can be comprised of sintered fibers, such as sintered metal fibers.

The aerosol generating component may comprise, for example, an electrically conductive thin layer of electrically resistive material, such as platinum, nickel, molybdenum, tungsten or tantalum, said thin layer being applied to a surface of the vaporizer by a PVD or CVD process, or any other suitable process. In this case, the aerosol generating component may comprise an electrically insulating material, for example of ceramic. Examples of suitable electrically resistive material include stainless steels, such as AISI 304 or AISI 316, and heating conductor alloys-in particular NiCr alloys and CrFeAl alloys (“Kanthal”), such as DIN material number 2,4658, 2,4867, 2,4869, 2,4872, 1,4843, 1,4860, 1,4725, 1,4765 and 1,4767.

As described above, the aerosol generating component may be formed from a sintered metal fiber material and may be in the form of a sheet. Material of this sort can be thought of a mesh or irregular grid, and is created by sintering together a randomly aligned arrangement or array of spaced apart metal fibers or strands. A single layer of fibers might be used, or several layers, for example up to five layers. As an example, the metal fibers may have a diameter of 8 to 12 μm, arranged to give a sheet of thickness 0.16 mm, and spaced to produce a material density of from 100 g/m² to 1500 g/m², such as from 150 g/m² to 1000 g/m², 200 g/m² to 500 g/m², or 200 to 250 g/m², and a porosity of 84%. The sheet thickness may also range from 0.1 mm to 0.2 mm, such as 0.1 mm to 0.15 mm. Specific thicknesses include 0.10 mm, 0.11 mm, 0.12 mm, 0.13 mm, 0.14 mm, 0.15 mm or 0.1 mm. Generally, the aerosol generating component has a uniform thickness. However, it will be appreciated from the discussion below that the thickness of the aerosol generating component may also vary. This may be due, for example, to some parts of the aerosol generating component having undergone compression. Different fiber diameters and thicknesses may be selected to vary the porosity of the aerosol generating component. For example, the aerosol generating component may have a porosity of 66% or greater, or 70% or greater, or 75% or greater, or 80% or greater or 85% or greater, or 86% or greater.

The aerosol generating component may form a generally flat structure, comprising first and second surfaces. The generally flat structure may take the form of any two dimensional shape, for example, circular, semi-circular, triangular, square, rectangular and/or polygonal. Generally, the aerosol generating component has a uniform thickness.

A width and/or length of the aerosol generating component may be from about 1 mm to about 50 mm. For example, the width and/or length of the vaporizer may be from 1 mm, 2 mm, 3 mm, 4 mm, 5 mm, 6 mm, 7 mm, 8 mm, 9 mm or 10 mm. The width may generally be smaller than the length of the aerosol generating component.

Where the aerosol generating component is formed from an electrically resistive material, electrical current is permitted to flow through the aerosol generating component so as to generate heat (so called Joule heating). In this regard, the electrical resistance of the aerosol generating component can be selected appropriately. For example, the aerosol generating component may have an electrical resistance of 2 ohms or less, such as 1.8 ohms or less, such as 1.7 ohms or less, such as 1.6 ohms or less, such as 1.5 ohms or less, such as 1.4 ohms or less, such as 1.3 ohms or less, such as 1.2 ohms or less, such as 1.1 ohms or less, such as 1.0 ohm or less, such as 0.9 ohms or less, such as 0.8 ohms or less, such as 0.7 ohms or less, such as 0.6 ohms or less, such as 0.5 ohms or less. The parameters of the aerosol generating component, such as material, thickness, width, length, porosity etc. can be selected so as to provide the desired resistance. In this regard, a relatively lower resistance will facilitate higher power draw from the power source, which can be advantageous in producing a high rate of aerosolization. On the other hand, the resistance should not be so low so as to prejudice the integrity of the aerosol generator. For example, the resistance may not be lower than 0.5 ohms.

Planar aerosol generating components, such as heating elements, suitable for use in systems, devices and articles disclosed herein may be formed by stamping or cutting (such as laser cutting) the required shape from a larger sheet of porous material. This may include stamping out, cutting away or otherwise removing material to create openings in the aerosol generating component. These openings can influence both the ability for air to pass through the aerosol generating component and the propensity for electrical current to flow in certain areas.

As described above, in one aspect the present invention relates to a delivery system comprising a powered aerosol generating device and an aerosolizable material, wherein the aerosolizable material comprises at least one carboxylated active, and wherein the system is configured to provide for selective decarboxylation of the carboxylated active. In particular, the aerosol generating device comprises a power source, such as an electrical power source, a controller and at least one aerosol generator (such as a heater) arranged to aerosolize (heat) the aerosolizable material to form an inhalable aerosol, wherein the controller is configured to facilitate delivery of power to the aerosol generator (heater) at more than one power level.

In more detail, article 30 contains an aerosolizable material located within a first reservoir 38. The aerosolizable material contains at least one active, such as a cannabinoid in carboxylated form, such as CBDA. Upon actuation of the device, either via the button or puff sensor (not shown), power will be delivered to the heater 36 such that an aerosol will be formed. When controlled to do so by the user, the controller 28 delivers power to heater 36 at an “elevated” power level. This elevated power level results in the CBDA present in the aerosolizable material being increasingly decarboxylated in situ whilst being vaporized. The resulting aerosol therefore contains decarboxylated CBD derived from the CBDA, as well as any other cannabinoids that were aerosolized during the heating process (such as decarboxylated cannabinoids already present in the aerosolizable material).

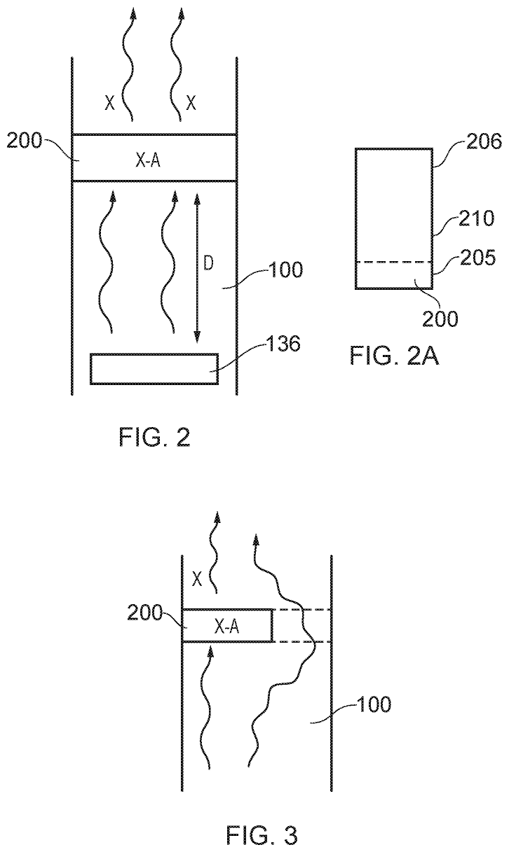

Referring now to FIGS. 2 and 2A, there is described a schematic view of part of an airflow channel through an aerosol delivery system as described herein. In particular, FIGS. 2 and 2A refer to another embodiment of the invention whereby a first aerosolizable material and a second aerosolizable material are stored at different locations within the system heater 136 is shown being located in the airflow channel 100. The heater 136 is fed with a first aerosolizable material in a manner as described herein (e.g. via a wick, not shown). During use, power is delivered from the power source of the device to the heater 136 such that an aerosol is formed from the first aerosolizable material. This first aerosol travels downstream within channel 100 until it encounters substrate 200 (which may be formed from a capillary material, such as cotton, cellulose acetate or some other porous material). Substrate 200 is loaded with a second aerosolizable material comprising at least one active (such as a cannabinoid) in carboxylated form (X-A—where X relates to the active, and A relates to a carboxyl group of the active). The substrate 200 completely spans the airflow channel 100, but due to its porous nature the first aerosol is permitted to pass through the substrate 20 such that heat from the first aerosol facilitates decarboxylation of X-A to form a second aerosol comprising the decarboxylated form of the active, such as the cannabinoid (denoted as X in FIG. 2). Substrate 200 is located a distance, D, from the heater 136. It is possible for this distance D to be varied so as to vary the impact temperature of the aerosol as it encounters the substrate. This variation can be achieved by employing substrate 200 in a removable segment 210 (shown in FIG. 2A) which can be positioned at various locations within the airflow channel as desired by the user. The removable segment 210 will typically contain a downstream end 205 where the substrate 200 is generally located, and an upstream end 206 which forms a mouthpiece for the user. The substrate 200 can be replaceable within the removable segment 210 so as to facilitate changing the substrate 200 from time to time. Removable segment 210 may be somewhat tapered so as to facilitate an interference fit when inserted into channel 100.

FIG. 3 refers to another embodiment whereby the substrate 200 does not completely span the airflow channel 100. This arrangement can be achieved by providing a segregated area within the downstream end 205 of the removable segment 210 such that the first aerosols through all segments, yet only some of the segments contain a substrate 200. Such an approach essentially dilutes the interaction between the first aerosol and the substrate leading to a second aerosol with relatively reduced levels of the decarboxylated active, such as a cannabinoid.

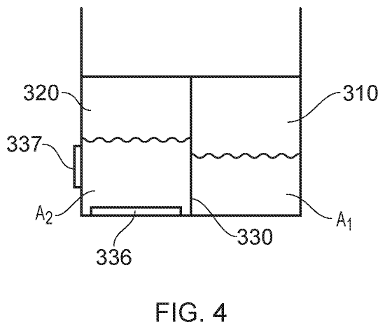

FIG. 4 refers to another embodiment of the invention whereby a first aerosolizable material and a second aerosolizable material are stored at different locations within the system. In particular, there is shown schematically part of an aerosol provision system where a first reservoir 310 and a second reservoir 320 are located. The first reservoir 310 contains a first aerosolizable material A1, and the second reservoir 320 contains a second aerosolizable material A2. The first and second reservoirs are fluidly connected. In this particular example, a membrane 330 separates the two reservoirs, but it might also be that a pump or other means for fluid transfer between the two reservoirs is provided. The second reservoir 320 may contain one or more heaters 336, 337. Heater 336 is provided internally. Heater 337 is provided externally. Both may be provided. Each heater is connected to the power source of the device (not shown) and controlled as described herein. Briefly, when controlled to do so by the user, heater 336 and/or heater 337 are supplied with power from the power source such that they heat aerosolizable material A2 to an elevated temperature as described herein. It should be understood that additional heaters may also be provided. Due to the elevated temperature, the carboxylated cannabinoid within aerosolizable material A2 will be decarboxylated. Over time, aerosolizable material A2 will become increasingly concentrated with decarboxylated active (such as a decarboxylated cannabinoid) and thus this decarboxylated cannabinoid can be supplied to the first reservoir for aerosolization by an aerosol generator (e.g. heater, not shown) into an inhalable aerosol. The cannabinoid present in each reservoir may be the same or different (independent of its carboxylated state) and may be any of the cannabinoids described herein. For example, the cannabinoid present in the first reservoir may be CBD, and the cannabinoid present in the second reservoir may be CBDA. Alternatively, the cannabinoid present in the first reservoir may be THC, and the cannabinoid present in the second reservoir may be CBDA.

### EXAMPLES

### Example 1

A study was executed to investigate the decarboxylation of CBDA to CBD in e-aerosol during vaping. An e-liquid sample containing 4.5% CBDA was formulated.

Aerosol was then generated from this e-liquid using an “ePod” (available at https://www.vuse.com/gb/en/e-cigarette-devices/epod-devices).

CBD measurements on e-liquids and e-aerosols were performed to measure the change in CBD levels before and after vaping. Measurements of CBD before and after aerosolization were carried out using a quantitative method based on liquid chromatography coupled with an ultraviolet diode array detector.

### Materials and Apparatus

Table 1 gives the unique identifiers of materials and apparatus used including the cannabinoid standards:

TABLE 1

| Item | Identifier |
|---|---|
| CBD (10 mg/mL in ethanol) | Sigma Aldrich, ELN ID: 21-0243 and 21-0244, |
| | Batch: BCCD7054 |
| CBD (1 mg/mL in methanol) | Cerilliant, ELN ID: 21-0316 and 21-0317, |
| | Batch: FE10071912 |
| CBDA (1 mg/mL in ACN) | Supelco, ELN ID: 21-0424, Batch: |
| | FE02202007 |
| CBDA | Purisys, ID: NDC#516342184, V-43, |
| | Batch: C16417 |
| Methanol (HPLC grade) | Fisher Scientific, CAS: 67-56-1, |
| | Batch: 2188005 |
| Propylene glycol | Sigma Aldrich, ELN ID: 19-0154, |
| | Batch: MKCH2860 |
| Glycerol | Sigma Aldrich, ELN ID: 21-0223, |
| | Batch: BCCF1935 |
| Device | ePod - |
| | https://www.vuse.com/gb/en/e-cigarette-devices/epod-devices |

Preparation of e-Liquid

10.4 g of 4.5% CBDA e-liquid was prepared (Propylene glycol, 7.0250 g; Glycerol, 2.9468 g; CBDA, 0.4659 g). The e-liquid was stored in a scintillation vial, covered in tin foil to exclude light. The e-liquid was then vortex mixed and stored in a cool dry environment overnight where it could homogenise to be ready for aerosolization.

### Aerosol Analysis

The day after the initial samples were prepared, sampling of the formulated e-liquid commenced. The e-liquid was split amongst 3 ePod cartomizers (1.2 mL each). Following the filling, the cartomizers were left to stand for 1 hour whilst stored away from light. Aerosol generation was carried out on a Borgwaldt 20 port puffing engine LM20e. Fully charged devices and their respective cartomizers (preweighed) were connected to pad holders containing a Cambridge filter pad (CFP) (pre-weighed) at a 15° device angle.

For the aerosol generation, a CRM81 puffing regime (55 mL of 3-second puff duration with a 30-second puff interval) was utilized. 4 puff blocks consisting of 20 puffs each was performed. Each of these puff blocks was collected on the same pad. The device mass loss (DML) was measured in between each puff block to estimate the trajectory for ACM generation. There was approximately a 4-minute interval in between puff blocks to try and produce more representative vaping samples, as opposed to 80 consecutive puffs.

Finally, following the completion of all the puff blocks, devices and pad holders were weighed to calculate the aerosol collected mass (ACM) and device mass loss (DML), which are shown in Table 2. Note, in between puff blocks, pad holders were not weighed, only devices.

TABLE 2

| Sample | Puff Block | DML (mg) | ACM (mg) |
|---|---|---|---|
| ePod replicate 1 | 1 | 123 | — |
| | 2 | 105 | |
| | 3 | 121 | |
| | 4 | 156 | |
| | Total | 505 | 506 |
| | SD | 21.4 | — |
| ePod replicate 2 | 1 | 114 | |
| | 2 | 107 | |
| | 3 | 114 | |
| | 4 | 135 | |
| | Total | 470 | 478 |
| | SD | 12.2 | — |
| ePod replicate 3 | 1 | 112 | |
| | 2 | 113 | |
| | 3 | 110 | |
| | 4 | 105 | |
| | Total | 440 | 451 |
| | SD | 3.7 | — |

Following the aerosol generation and collection, CFP's were removed from the pad holders, folded in half and the side of the pad holder not facing the device was used to wipe the inside of the pad holder. These were then transferred to conical flasks and 20 mL of methanol was added to each flask. This was done to aim for an approximate 1 mg/mL CBD concentration assuming 100% decarboxylation. The flasks were covered in foil to exclude light and shaken at 150 rpm for 45 minutes.

Following shaking, extracts of the solution were syringe-filtered (Merck Millipore Milex-GV PVDF 0.22 μm filters) into LC vials. No further dilutions were required as the 1 mg/mL target was reached with the extraction solution dilutions of the ACM. The solutions were crimped, and vortex mixed for 5 seconds before loading onto the instrument. Whilst vialing the samples for HPLC-UVDAD injection, every effort was made to reduce the exposure of light on the pad extracts. Foil was wrapped around the flasks at all points possible.

### E-Liquid Analysis

Three replicates of the CBDA formulation were produced. In triplicate, 220 μL of e-liquid was volumetrically dispensed and weighed out. The e-liquids were then diluted in 10 mL of methanol and mixed thoroughly through gentle inversions followed by 5 seconds on the vortex mixer. The samples were then syringe-filtered (Merck Millipore Milex-GV PVDF 0.22 μm filters) into LC vials, crimped and vortex mixed for a further 5 seconds. These samples were then loaded onto the instrument for same-day injection.

To demonstrate the accuracy of the method, one replicate of diluted e-liquid and one replicate of diluted aerosol were fortified by the addition of know concentrations of CBD as shown in Table 3:

TABLE 3

| | Volume of Sample | Volume of 1 mg/mL | Fortification Level |
|---|---|---|---|
| Sample | (μL) | CBD (μL) | (mg/mL) |
| e-aerosol_1_epod | 900 | 100 | 0.1 |
| e-liquid_1 | 900 | 100 | 0.1 |

### HPPL-UVDAD Analysis

All calibration standards, calibration check standards, blanks, test samples and fortified samples were analysed by HPLC-UVDAD using an Agilent 1260 Liquid Chromatograph with autosampler, column oven, and UV analysis via Diode array detector (DAD), and 1260 binary pumps.

One separate sequence was run. Before the run was initiated, LC lines were flushed using freshly prepared mobile phases. After the lines were flushed, the mobile phase was diverted through the column. This was left to purge for 1 hour before the injections began. Agilent OpenLab CDS ChemStation Edition was used to run sequences and data process. All the chromatograms were reviewed for retention time, peak shape and to confirm suitable peak integration by the software.

### Results

Six calibration standards were injected as part of the sequence, spanning a range of 0.05-2 mg/mL of CBD (shown in Table 4).

TABLE 4

| Calibration standard level | [CBD] (mg/mL) |
|---|---|
| 1 | 0.05 |
| 2 | 0.1 |
| 3 | 0.5 |
| 4 | 1 |
| 5 | 1.5 |
| 6 | 2 |

The accuracy of this calibration curve was monitored throughout the run by injection of calibration check standards and fortified samples. The calibration curve demonstrated good accuracy throughout the run (see Table 5). Additionally, a good accuracy is observed with the fortified e-aerosol.

TABLE 5

| Sample | Accuracy (%) |
|---|---|
| CBD Cal Check Injection 1 | 100 |
| CBD Cal Check Injection 2 | 99 |
| Fortified e-aerosol | 100 |
| Fortified e-liquid | n/a |

Generation of a quantitative result for the fortified e-liquid was not possible as the native CBD amount in the unfortified e-liquid was <LOQ (limits of quantification).

The measured levels of CBD in the e-liquids (mg/g) is shown in Table 6.1

TABLE 6.1

| E-liquid replicate | Measured concentration of CBD (mg/g) |
|---|---|
| 1 | <LOQ |
| 2 | <LOQ |
| 3 | <LOQ |

The measured levels of CBD in aerosol replicates are shown in Table 6.2 (mg CBD per gram of e-liquid).

TABLE 6.2

| Device and Replicate | Measured concentration of CBD (mg/g) |
|---|---|
| ePod replicate 1 | 20.7 |
| ePod replicate 2 | 20.5 |
| ePod replicate 3 | 23.3 |
| eTank Pro replicate 1 | 28.5 |

The results presented in Table 6.1 show that levels of CBD prior to aerosolization were below the LOQ. Hence, any CBD found in the aerosol must be due to the aerosolization process. To estimate the decarboxylation percentage, the following calculations were required. Initially the concentration of CBDA in e-liquid is known as it has been formulated in the lab. This concentration was used to calculate the concentration of CBD assuming a 100% decarboxylation. A ratio of these compounds (CBDA and CBD) was required. This is due to their different molecular masses.

$$Ratio of CBD from CBDA = ( 314.469 {gmol}^{- 1} \div 358.478 {gmol}^{- 1} ) = 0.877 \qquad Eq 1$$

Using this ratio and the initial CBDA concentration the expected concentration of CBD was calculated (assuming 100% decarboxylation). This expected concentration also assumes a 100% purity of CBDA used for formulation, and no chemical or physical losses of CBDA in the e-liquid between formulation and e-aerosol collection.

$$Expected concentration of CBD = ( 0.877 \times CBDAe - liquid concentration ) = 39.5 \frac{mg}{g} \qquad Eq 2$$

Finally, values from Table 6.2 were divided by 39.5 mg/g to give the values shown in Table 7.

The determined decarboxylation is shown in Table 7:

TABLE 7

| Sample | Estimated Decarboxylation (%) |
|---|---|
| ePod replicate 1 | 52 |
| ePod replicate 2 | 52 |
| ePod replicate 3 | 59 |

The results indicate initial levels of CBD in the CBDA e-liquid are bellow limits of quantitation. Upon aerosolization quantifiable levels of CBD are presented. Hence, decarboxylation of CBDA to CBD is present in the tested vaping system.

### Example 2

A further test was conducted to investigate the impact of various usage conditions on the conversion process.

The e-liquid used for this example contained 5.61% w/w CBDA (the balance of the formulation comprising 70% w/w propylene glycol and 24.39% w/w glycerol).

The study device is a commercially available e-cigarette device (Kangertech Evod Variable Voltage (VV) 1300 mah with a Kanger EVOD clearomizer+MT32 synthetic silica wick 1.8Ω coils).

The device is button operated and voltage/power is varied by rotating the base of the device. This device was used as it enables both power and puff duration to be varied within the boundaries of conventional vaping behavior. All tanks are maintained at a minimum of 50% volume of the maximum liquid level and are consistent between experiments. The airflow through the device was fixed.

### Power Variation:

5 W
8 W
12 W

### Puffing Variation:

Low—55 mL puff volume, 3 second puff duration
Medium—55 mL puff volume, 4 second puff duration
High—55 mL puff volume, 5 second puff duration

The analysis of CBDA, CBD employed an LC-DAD-MS approach (Agilent 1260 Liquid Chromatograph with autosampler, column oven, UV analysis via Diode array detector (DAD), quaternary pumps (600 bar); Varian/Agilent MS 500 Ion Trap equipped with Electrospray Ion source. The stationary phase used was an Agilent SB C18 4.6×100 with particle size of 1.7 micron).

Reference compounds were obtained from Sigma Aldrich. Samples were diluted in mixture acetone/ethanol 1:1 on the basis of the initial concentration to reach a final CBD/CBDA concentration of about 100 microgram/mL.

For the detection of the cannabinoids, the DAD detector was set at 280 nm and spectra will be collected in the range 200-400 nm. Mass spectra was acquired in positive ion mode (for the analysis of neutral cannabinoids) and negative ion mode (for the analysis of acidic forms). Calibration curves were obtained plotting area (at 280 nm) versus concentration (ug/mL) in the range 500-5 ug/mL. Limit of detection by DAD at 280 nm is 0.5 micrograms/mL. Mass spectrometry was used as confirmation of the structure of the detected compounds as well as to analyse lower amount of trace compounds. Calibration curves were obtained also in Mass spectrometry plotting area of the ion specie ([M+H]+ for neutral forms [M−H]− for acidic forms) versus concentration.

Reference standards used for quantification of CBDA and CBD (product code=PHL85705 https://www.sigmaaldrich.com/GB/en/product/supelco/ph185705), and for CBDA (product code=39961 https://www.sigmaaldrich.com/GB/en/product/sial/39961)

The results of the evaluation of the power and puffing variations are shown in Table 8:

TABLE 8

| | | | | | | | av. CBDA | av. CBD | | |
|---|---|---|---|---|---|---|---|---|---|---|
| Liquid | Puff | Power | | | DML | av. DML | (mg) in total | (mg) in total | conversion | conversion |
| composition | duration | output | | Replicates | (g) | (mg) | ACM | ACM | ratio | ratio/DML |
| E-LIQUID | Low intensity | 5 | W | Pod 1 | 0.2258 | 227.7333 | 1.33 | 2.28 | 63.16% | 63.16% |
| 5.61:70:24.39:0 | regimen: 3 sec | | | Pod 2 | 0.2233 | | | | | |
| (CBDA:PG:VG:H2O) | puff | | | Pod 3 | 0.2341 | | | | | |
| | | 8 | W | Pod 1 | 0.3313 | 328.53333 | 4.84 | 8.03 | 62.39% | 62.39% |
| | | | | Pod 2 | 0.3102 | | | | | |
| | | | | Pod 3 | 0.3441 | | | | | |
| | | 12 | W | Pod 1 | 0.3508 | 388.7 | 2.94 | 11.7 | 79.92% | 79.92% |
| | | | | Pod 2 | 0.4278 | | | | | |
| | | | | Pod 3 | 0.3875 | | | | | |
| | Mid intensity | 5 | W | Pod 1 | 0.4302 | 383.56667 | 3.43 | 12.89 | 78.98% | 78.98% |
| | regimen: 4 sec | | | Pod 2 | 0.3679 | | | | | |
| | puff | | | Pod 3 | 0.3526 | | | | | |
| | | 8 | W | Pod 1 | 0.5810 | 577.8 | 6.03 | 22.32 | 78.73% | 78.73% |
| | | | | Pod 2 | 0.5730 | | | | | |
| | | | | Pod 3 | 0.5794 | | | | | |
| | | 12 | W | Pod 1 | 0.5980 | 613.76667 | 4.46 | 19.15 | 81.11% | 81.11% |
| | | | | Pod 2 | 0.6278 | | | | | |
| | | | | Pod 3 | 0.6155 | | | | | |
| | High intensity | 5 | W | Pod 1 | 0.4398 | 478.46667 | 3.66 | 18.43 | 83.43% | 83.43% |
| | regimen: 5 sec | | | Pod 2 | 0.4505 | | | | | |
| | puff | | | Pod 3 | 0.5451 | | | | | |
| | | 8 | W | Pod 1 | 0.7254 | 689.33333 | 8.14 | 25.29 | 75.65% | 75.65% |
| | | | | Pod 2 | 0.6765 | | | | | |
| | | | | Pod 3 | 0.6661 | | | | | |
| | | 12 | W | Pod 1 | 0.7994 | 828.4 | 3.33 | 38.9 | 92.11% | 92.11% |
| | | | | Pod 2 | 0.8353 | | | | | |
| | | | | Pod 3 | 0.8505 | | | | | |

It is apparent that increasing the power leads to a greater amount of CBD in the aerosol. In particular, the relative percentage of CBD (compared to CBDA) in the aerosol is generally higher for higher power settings. Moreover, longer puffs generally also lead to an increased relative percentage of CBD (compared to CBDA) in the aerosol.

### Example 3

A further example was conducted in the same fashion as for Example 2, but using a formulation which also contained water (CBDA 5.61% w/w; propylene glycol, 70% w/w; glycerol, 21.39% w/w; water, 3% w/w. The results for this example are shown in Table 9.

As can be observed, at lower powers, adding water to the formulation resulted in an increase in relative percentage of CBD (compared to CBDA) than in the formulations where no water was present. Using a formulation comprising water therefore enables higher CBD percentages in the aerosol at lower powers.

TABLE 9

| | | | | | | av. CBDA | av. CBD | | |
|---|---|---|---|---|---|---|---|---|---|
| Puff | Power | | | DML | av. DML | (mg) in total | (mg) in total | conversion | conversion |
| duration | output | | Replicates | (g) | (mg) | ACM | ACM | ratio | ratio/DML |
| Low | 5 | W | Pod 1 | 1.0460 | 191.93 | 0.74 | 4.56 | 86.04% | 86.04% |
| intensity | | | Pod 2 | 1.0037 | | | | | |
| regimen: | | | Pod 3 | 1.0075 | | | | | |
| 3 sec puff | 8 | W | Pod 1 | 1.0074 | 364.17 | 3.18 | 10.80 | 77.25% | 77.25% |
| | | | Pod 2 | 1.0180 | | | | | |
| | | | Pod 3 | 1.0235 | | | | | |
| | 12 | W | Pod 1 | 1.0304 | 400.67 | 3.31 | 11.80 | 78.09% | 78.09% |
| | | | Pod 2 | 1.0081 | | | | | |
| | | | Pod 3 | 1.0045 | | | | | |
| Mid | 5 | W | Pod 1 | 0.9945 | 322.37 | 0.54 | 8.68 | 94.14% | 94.14% |
| intensity | | | Pod 2 | 1.0354 | | | | | |
| regimen: | | | Pod 3 | 0.9321 | | | | | |
| 4 sec puff | 8 | W | Pod 1 | 1.0035 | 438.43 | 4.92 | 19.40 | 79.77% | 79.77% |
| | | | Pod 2 | 1.0199 | | | | | |
| | | | Pod 3 | 1.0349 | | | | | |
| | 12 | W | Pod 1 | 1.0388 | 610.80 | 5.02 | 32.20 | 86.51% | 86.51% |
| | | | Pod 2 | 1.0425 | | | | | |
| | | | Pod 3 | 1.0562 | | | | | |

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A delivery system comprising a powered aerosol generating device and an aerosolizable material, wherein the powered aerosol generating device comprises a power source and a controller, and the system comprises a first aerosolizable material and a second aerosolizable material, wherein the second aerosolizable material comprises at least one carboxylated active and is stored in the system separately from the first aerosolizable material.

2. The system according to claim 1, wherein the second aerosolizable material is stored in a second reservoir which is separate from a first reservoir in which the first aerosolizable material is located.

3. The system according to claim 2, wherein one or more heaters are configured to heat the second aerosolizable material in the second reservoir.

4. The system according to claim 3, wherein the one or more heaters are selected from one or more internal heaters in contact with the second aerosolizable material and/or one or more external heaters which are not in contact with the second aerosolizable material.

5. The system according to claim 3, wherein the one or more heaters are configured to heat the second aerosolizable material to a temperature above ambient, but below the temperature at which significant vaporization of the second aerosolizable material would take place.

6. The system according to claim 5, wherein the temperature to which the second aerosolizable material is heated is between 50° C. and 150° C.

7. The system according to claim 3, wherein the second aerosolizable material is heated for a sustained period of time.

8. The system according to claim 2, wherein the first and second reservoirs are fluidly connected.

9. The system according to claim 8, wherein the user can control the extent to which transfer of the second aerosolizable material to the first reservoir occurs.

10. The system according to claim 9, wherein a pump controls the transfer of a particular quantity of second aerosolizable material when controlled to do so by the user.

11. The system according to claim 9, wherein a button, switch, touchpad or the like is used to control the transfer of the second aerosolizable material to the first reservoir.

12. The system according to claim 9, wherein the controller is configured to initiate transfer of a particular quantity of second aerosolizable material to the first reservoir according to a particular schedule.

13. The system according to claim 1, wherein the system comprises an aerosol generator, such as a heater, arranged to convert the first aerosolizable material to an aerosol.

14. The system according to claim 13, wherein the second aerosolizable material is located on a substrate which is located within the system such that, in use, aerosol from the first aerosolizable material contacts the substrate.

15. The system according to claim 14, wherein the substrate is a porous substrate.

16. The system according to claim 1, wherein the at least one carboxylated active is a cannabinoid, such as cannabidiolic acid (CBDA).

17. The system according to claim 1, wherein the second aerosolizable material comprises one or more further actives, such as cannabinoids, wherein the one or more further actives are in the decarboxylated form when in the aerosolizable material.

18. The system according to claim 17, wherein the one or more further actives in the decarboxylated form is cannabidiol (CBD).

19. The system according to claim 17, wherein the ratio of the at least one active present in the carboxylated form to the one or more further actives in the decarboxylated form is from 99:1 to 1:99.

20. The system according to claim 1, wherein the first aerosolizable material comprises the one or more actives in the decarboxylated form when in the first aerosolizable material.

21. The system according to claim 1, wherein the aerosolizable material further comprises a carrier constituent and optionally one or more flavours.

22. The system according to claim 21, wherein the carrier constituent comprises one or more of propylene glycol and glycerol.
